(19) United States
(12) Patent Application Publication
(10) Pub. No.: US 2023/0111533 A1
(43) Pub. Date: Apr. 13, 2023

(54) PREPARATION METHOD OF ION CATALYST MATERIAL FOR PET CHEMICAL RECYCLING AND PET CHEMICAL RECYCLING METHOD

(71) Applicant: NAN YA PLASTICS CORPORATION, Taipei (TW)

(72) Inventors: Te-Chao Liao, Taipei (TW); Sen-Huang Hsu, Taipei (TW); Yu-lin Li, Taipei (TW)

(73) Assignee: NAN YA PLASTICS CORPORATION, Taipei (TW)

(21) Appl. No.: 17/826,195

(22) Filed: May 27, 2022

(30) Foreign Application Priority Data
Oct. 7, 2021 (TW) 110137404

(51) Int. Cl. B01J 37/02; B01J 27/10; B01J 27/128; B01J 27/138; B01J 27/32; B01J 31/18; B01J 14/00; C08J 11/10

(52) U.S. Cl. CPC B01J 37/0213; B01J 27/10; B01J 27/128; B01J 27/138; B01J 27/32; B01J 31/1815; B01J 37/0209; B01J 14/005; C08J 11/10; C08J 2367/03

(57) ABSTRACT

Provided are a preparation method of an ion catalyst material for PET chemical recycling and a PET chemical recycling method. The preparation method of an ion catalyst material for PET chemical recycling includes the following. A metal chloride is added to an alkylimidazole-chloride ionic liquid to form a bisalkylimidazole-metal tetrachloride ionic liquid that is grafted on a porous carrier.

### CROSS-REFERENCE TO RELATED APPLICATION

This application claims the priority benefit of Taiwan application serial no. 110137404, filed on Oct. 7, 2021. The entirety of the above-mentioned patent application is hereby incorporated by reference herein and made a part of this specification.

### BACKGROUND

### Technical Field

The disclosure relates to a preparation method of an ion catalyst material for PET chemical recycling and a PET chemical recycling method, and in particular, to a preparation method of an ion catalyst material for PET chemical recycling and a PET chemical recycling method which achieve decolorization and depolymerization and facilitate recycling.

### Related Art

Polyethylene terephthalate (PET) is one of the most widely used plastics and may be used in rigid packaging materials for beverages and food, as well as textile fabrics. In view of the global trend of environmental protection and pollution prevention, many governments require both public agencies and private businesses to use recycled PET products, and major international brands have set targets for using recycled materials. These measures have promoted the development of PET recycling technology.

With the increasing complexity of commercially available PET packaging materials and the widespread use of PET, recycling operations have become more and more complex. Thus, chemical recycling for recycling and depolymerization of PET waste plastics have received attention. Recently, the closed-loop chemical recycling technology for PET has developed rapidly around the globe. Techniques such as solvent-assisted chemical degradation, biodegradation, and microwave-assisted chemical degradation have reached the trial mass production stage, in which different types of PET waste plastics, such as packaging materials and textile fabrics, can be processed. However, in most of the current PET chemical recycling technologies, depolymerization and decolorization need to be separately performed. In addition, recycling of a catalyst is not easy, thereby significantly increasing the cost.

Based on the above, it has become a hot research topic to develop a chemical recycling technique for the recycling and depolymerization of PET waste plastics to make it possible to perform depolymerization and decolorization at the same time and to recycle a catalyst.

### SUMMARY

The disclosure provides a preparation method of an ion catalyst material for PET chemical recycling and a PET chemical recycling method, in which decolorization and depolymerization can be achieved and recycling can be facilitated.

A preparation method of an ion catalyst material for PET chemical recycling according to the disclosure includes the following. A metal chloride is added to an alkylimidazole-chloride ionic liquid to form a bisalkylimidazole-metal tetrachloride ionic liquid that is grafted on a porous carrier.

In an embodiment of the disclosure, the metal chloride includes iron chloride, zinc chloride, or cobalt chloride.

In an embodiment of the disclosure, the porous carrier includes nano iron oxide, silicon oxide, or activated carbon.

In an embodiment of the disclosure, a ratio of the metal chloride to the alkylimidazole-chloride ionic liquid is 1 to 2.

In an embodiment of the disclosure, the porous carrier has a graft rate of 6% to 10%.

A PET chemical recycling method according to the disclosure includes the following. A PET chemical recycling ion catalyst material prepared by the above-mentioned preparation method of an ion catalyst material for PET chemical recycling is added to PET waste and ethylene glycol, followed by heating and stirring to obtain depolymerized and decolorized bis(2-hydroxyethyl)terephthalate (BHET). Then, the ion catalyst material for PET chemical recycling is filtered and recycled.

In an embodiment of the disclosure, the PET waste includes a packaging material or a textile fabric.

In an embodiment of the disclosure, a ratio of the PET waste to the ethylene glycol is 1:3 to 1:6.

In an embodiment of the disclosure, an addition amount of the ion catalyst material for PET chemical recycling is 0.5% to 2%.

In an embodiment of the disclosure, a heating temperature is 180° C. to 210° C., a stirring speed is 100 rpm to 150 rpm, and stirring time is 3 hours to 6 hours.

Based on the above, in the preparation method of an ion catalyst material for PET chemical recycling according to the disclosure, a porous material is used as a carrier so as to improve the decolorization ability with respect to PET waste (especially a textile fabric), and a bisalkylimidazole-metal tetrachloride ionic liquid is used so as to improve the depolymerization ability. In this way, in the disclosure, decolorization and depolymerization can be achieved and recycling can be facilitated by a simple manufacturing process.

### DESCRIPTION OF EMBODIMENTS

Hereinafter, embodiments of the disclosure will be described in detail. However, these embodiments are illustrative, and the disclosure is not limited thereto.

In the present specification, a range represented by “a numerical value to another numerical value” is a schematic representation for avoiding listing all of the numerical values in the range in the specification. Therefore, the recitation of a specific numerical range covers any numerical value in the numerical range and a smaller numerical range defined by any numerical value in the numerical range, as is the case with any numerical value and a smaller numerical range thereof in the specification.

The disclosure provides a preparation method of an ion catalyst material for PET chemical recycling. The preparation method includes the following. A metal chloride is added to an alkylimidazole-chloride ionic liquid to form a bisalkylimidazole-metal tetrachloride ionic liquid that is grafted on a porous carrier.

In the present embodiment, the metal chloride may include iron chloride, zinc chloride, or cobalt chloride. However, the disclosure is not limited thereto. A ratio of the metal chloride to the alkylimidazole-chloride ionic liquid is 1 to 2. The porous carrier may include nano iron oxide, silicon oxide, or activated carbon. However, the disclosure is not limited thereto. The porous carrier has a graft rate of about 6% to 10%.

The disclosure also provides a PET chemical recycling method including the following. A PET chemical recycling ion catalyst material prepared by the above-mentioned preparation method of an ion catalyst material for PET chemical recycling is added to PET waste and ethylene glycol, followed by heating and stirring to obtain depolymerized and decolorized bis(2-hydroxyethyl)terephthalate (BHET). Then, the ion catalyst material for PET chemical recycling is filtered and recycled.

In the present embodiment, the PET waste may include a packaging material or a textile fabric. However, the disclosure is not limited thereto. A ratio of the PET waste to the ethylene glycol is, for example, 1:3 to 1:6. An addition amount of the ion catalyst material for PET chemical recycling is, for example, 0.5% to 2%. A heating temperature is, for example, 180° C. to 210° C., a stirring speed is, for example, 100 rpm to 150 rpm, and stirring time is, for example, 3 hours to 6 hours.

By the PET chemical recycling method according to the disclosure, in an experimental example for a textile fabric, after being subjected to depolymerization, BHET purification, and polymerization, the textile fabric was subjected to gel permeation chromatography (GPC) testing, in which depolymerization efficiency was measured as 93%, and chromatic values were measured as an L value of 85 to 90, an a value of −1 to 1, and a b value of 1 to 4.

In summary, in the preparation method of an ion catalyst material for PET chemical recycling according to the disclosure, a porous material is used as a carrier so as to improve the decolorization ability with respect to PET waste (especially a textile fabric), and a bisalkylimidazole-metal tetrachloride ionic liquid is used so as to improve the depolymerization ability. In this way, in the disclosure, decolorization and depolymerization can be achieved and recycling can be facilitated by a simple manufacturing process. Accordingly, the problems of the related art, such as that depolymerization and decolorization need to be separately performed and recycling of a catalyst is not easy, can be effectively eliminated, and the cost can be reduced.

What is claimed is:

1. A preparation method of an ion catalyst material for PET chemical recycling, comprising:
adding a metal chloride to an alkylimidazole-chloride ionic liquid to form a bisalkylimidazole-metal tetrachloride ionic liquid that is grafted on a porous carrier.

2. The preparation method of an ion catalyst material for PET chemical recycling according to claim 1, wherein the metal chloride comprises iron chloride, zinc chloride, or cobalt chloride.

3. The preparation method of an ion catalyst material for PET chemical recycling according to claim 1, wherein the porous carrier comprises nano iron oxide, silicon oxide, or activated carbon.

4. The preparation method of an ion catalyst material for PET chemical recycling according to claim 1, wherein a ratio of the metal chloride to the alkylimidazole-chloride ionic liquid is 1 to 2.

5. The preparation method of an ion catalyst material for PET chemical recycling according to claim 1, wherein the porous carrier has a graft rate of 6% to 10%.

6. A PET chemical recycling method, comprising:
adding a PET chemical recycling ion catalyst material prepared by the preparation method of an ion catalyst material for PET chemical recycling according to claim 1 to PET waste and ethylene glycol, followed by heating and stirring to obtain depolymerized and decolorized bis(2-hydroxyethyl)terephthalate (BHET); and
filtering and recycling the ion catalyst material for PET chemical recycling.

7. The PET chemical recycling method according to claim 6, wherein the PET waste comprises a packaging material or a textile fabric.

8. The PET chemical recycling method according to claim 6, wherein a ratio of the PET waste to the ethylene glycol is 1:3 to 1:6.

9. The PET chemical recycling method according to claim 6, wherein an addition amount of the ion catalyst material for PET chemical recycling is 0.5% to 2%.

10. The PET chemical recycling method according to claim 6, wherein a heating temperature is 180° C. to 210° C., a stirring speed is 100 rpm to 150 rpm, and stirring time is 3 hours to 6 hours.
